(19) United States
(12) Patent Application Publication
(10) Pub. No.: US 2016/0184076 A1
(43) Pub. Date: Jun. 30, 2016

(54) Implantable Endoprosthesis for Aortic Aneurysm

(71) Applicant: CORDIS CORPORATION, Fremont, CA (US)

(72) Inventors: Animesh CHOUBEY, Fremont, CA (US); David MAJERCAK, Livermore, CA (US)

(73) Assignee: CORDIS CORPORATION, Fremont, CA (US)

(21) Appl. No.: 14/587,526

(22) Filed: Dec. 31, 2014

(51) Int. Cl. A61F 2/07; A61F 2/962; A61F 2/856; A61F 2/89; A61F 2/844; A61F 2/852

(52) U.S. Cl. CPC A61F 2/07; A61F 2/844; A61F 2/852; A61F 2/856; A61F 2/89; A61F 2/962; A61F 2002/061

(57) ABSTRACT

Described are various embodiments of an improved endoprosthesis that includes at least one tubular graft section coupled to additional tubular graft sections which are then coupled to a tubular bifurcated main section. Various embodiments described and shown herein allow for a health care provider to design and select an appropriate AAA implant for AAA presentations other than an infrarenal AAA.

### BACKGROUND

An aneurysm is an abnormal dilation of a layer or layers of an arterial wall, usually caused by a structural defect due to hardening of the artery walls or other systemic defects such as aortic dissection due to high blood pressure. The widely accepted approach to treating an aneurysm in the abdominal aorta (i.e., an “abdominal aortic aneurysm” or “AAA”) is by surgical repair, involving replacing the aneurysmal segment with a prosthetic device. This surgery is a major undertaking, with associated high risks and with significant mortality and morbidity.

A typical surgical repair for AAA is performed by making an incision into the abdomen to allow the physician to access the aorta (FIG. 8A). Once the aorta is accessible, it may be clamped to allow the surgeon to cut open the aorta and suture one graft end proximal to the heart. The other end of the graft is sutured to the aorta at a location past the aneurysm. This allows the blood flow from the heart to bypass the weakened area of the aorta.

One alternative to the surgical repair is to use an endovascular procedure, i.e., catheter directed, techniques for the treatment of aneurysms, specifically for AAA. This has been facilitated by the development of vascular stents, which can and have been used in conjunction with standard or thin-wall graft material in order to create a stent-graft or endograft. The potential advantages of less invasive treatments have included reduced surgical morbidity and mortality along with shorter hospital and intensive care unit stays.

One concern with the use of an endograft (or endoprosthesis) for AAA is that most if not all AAA endoprosthesis are configured for presentation of AAA as an infrarenal AAA. As shown in FIG. 8A1, an infrarenal typically presents sufficient landing zones for the implant to achieve a tight seal between the inner surface of the vessel wall of the aorta and the outer surface of the endoprosthesis. Where the distance between the renal arteries and aneurysm (i.e., the “neck length”) is less than 15 mm, it is believed that complications may result from the use of an endoprosthesis designed for an infrarenal presentation. Thus, in the presentation of a neck length of less than 15 mm, a juxtarenal AAA (FIG. 8BII), pararenal AAA (FIG. 8BIII), or a suprarenal AAA (FIG. 8BIV), it is believed that complications would certainly result from the use of the existing AAA endoprosthesis.

Others in this field have attempted to overcome the drawbacks of existing AAA endoprosthesis by utilizing what is known in the field as the “fenestrated technique”. This technique relies on hand-made customized fenestrations to incorporate both the renal and superior mesenteric arteries into such bespoke endoprosthesis for juxtarenal to suprarenal AAAs. In one aspect of the fenestrated technique, a physician can make openings or fenestrations by hand to an off-the-shelf AAA implant. The drawbacks to physician modified fenestrated implants are that the implants are not FDA approved, requiring the physician to apply for a regulatory waiver and such fenestrated implants may take hours to make by the physician. To alleviate these drawbacks, manufacturers have provided customized fenestrated implant based on imaging of the aneurysm 6-12 weeks before the scheduled implant. However, one drawback to this technique is that a peculiar anatomy of the renal arteries may render the customized implant ineffective. For example, there may be an extra renal or hepatic artery involved, as well as renal arteries that are oriented upward. Additionally, the bespoke implants typically require a long-lead time by which time the anatomy of the AAA could have changed significantly resulting in branching arteries that do not align with the fenestrations. Even if the known implant could be modified during the day of the implant by the physician (to avoid the time lag issue for the customized implant noted earlier), such physician-modified-implant (as well as the custom-made implant) may still not be ideal due to angulation of the anatomy causing the custom fenestrations to shift from the ideal alignment with the branching arteries.

### SUMMARY OF THE DISCLOSURE

Accordingly, we have devised an implantable endoprosthesis overcomes the disadvantages in the bespoke fenestration in that a physician does not have to hand make a custom implant a few hours before the implantation procedure. And our invention overcomes the problems associated with an implant made by order weeks in advance before the actual AAA operation whereby the anatomy or the aneurysm may have changed during the time the implant was ordered and actually implanted. In brief, the invention provides for three key improvements: (1) ease of use in the simplification of deployment for one fenestration at a time; (2) in-situ alignment of each opening to the targeted branching artery resulting in improved clinical outcomes; and (3) the overall profile of the endoprosthesis is ultra-low (i.e., less than 16 F for large native artery and in most cases, less than 12 French) because each portion of the endoprosthesis is smaller while requiring only one extra guidewire lumen.

Thus, our inventive device includes a first portion, second portion and retention structure for one (or even both of the first and second portions) that heretofore was not available in the art. The first portion extends along a longitudinal axis and includes a generally tubular graft defining a generally circular opening disposed about the longitudinal axis. The first portion includes a second end defining a generally elliptical opening about the longitudinal axis. The second portion extends along the longitudinal axis and having a first end including a generally tubular graft defining a generally elliptical opening with respect to the longitudinal axis to allow the second end of the first portion to extend into the generally elliptical opening of the second portion. The second portion has a bifurcation that extends into two limbs extending along the longitudinal axis.

We have also devised yet a variation on this endoprosthesis that includes a first, second and third portions along with a retention structure that can be utilized with one or the entire first through third portions. In particular, the first portion extends along a longitudinal axis, and has a first end defining a generally circular opening orthogonal to the longitudinal axis with retention barbs coupled to a retention structure connected to the generally circular opening. The first portion includes a second end defining a generally elliptical opening about the longitudinal axis. The second portion extends along the longitudinal axis and has a first end defining a generally elliptical opening with respect to the longitudinal axis to allow the second end of the first portion to extend into the generally elliptical opening of the second portion. The second portion has a second end defining a generally circular opening orthogonal to the longitudinal axis. The third portion extends along the longitudinal axis and has a first end defining a generally elliptical opening with respect to the longitudinal axis to allow the second end of the second portion to telescope thereto with respect to the generally elliptical opening of the third portion. The third portion has a bifurcation that extends into two limbs extending along the longitudinal axis.

In yet another variation, an endovascular implant is provided that includes three generally tubular portions with a retention structure for the first and third tubular portions. In particular, the first portion extends along a longitudinal axis and has a first end defining a first generally circular opening orthogonal to the longitudinal axis with retention barbs coupled to a retention structure connected to the generally circular opening. The first portion includes a second end defining a generally elliptical opening about the longitudinal axis; a second portion extending along the longitudinal axis, the second portion having a first end defining a generally elliptical opening with respect to the longitudinal axis to allow the second end of the first portion to extend into the generally elliptical opening of the second portion, the second portion having a second end defining a second generally circular opening orthogonal to the longitudinal axis. The third portion extends along the longitudinal axis and has a first end defining a third generally circular opening orthogonal to the longitudinal axis to allow the second end of the second portion to telescope with respect thereto the first end of the third portion, the first end of the third portion having retention members coupled to the third generally circular opening. The third portion includes a bifurcation that extends into two limbs extending along the longitudinal axis.

In addition to the embodiments described above, other features recited below can be utilized in conjunction therewith. For example, each of the first, second and third portions comprises a plurality of stent hoops spaced along the longitudinal axis and attached to a graft material to define a stent graft composite implant, each of the stent hoops having a sinusoidal configuration disposed about the longitudinal axis with apices spaced apart along the longitudinal axis; one apex of one stent hoop is disposed between two apices of another stent hoop; the generally tubular graft comprises a synthetic material selected from a group consisting of nylon, ePTFE, PTFE, Dacron and combinations thereof; the plurality of stent hoops are disposed on the inside surface of the stent-graft; a first peripheral opening is formed through the graft material about the longitudinal axis of the first portion proximate the first end and so that the first peripheral opening faces a mesenteric artery when the implant is deployed in an abdominal artery; a second peripheral opening is formed through the graft material about the longitudinal axis of the first portion so that the second peripheral opening faces a renal artery when the implant is deployed in the abdominal artery to allow fluid communication from the renal artery to the second; a third peripheral opening is formed through the graft material about the longitudinal axis of the second portion so that the third peripheral opening faces another renal artery when the implant is deployed in the abdominal artery to allow fluid communication from the renal artery to the third peripheral opening; the first portion is radially adjustable with respect to the second portion so that the first peripheral opening on the first portion is generally diametrical to the first peripheral opening on the second portion and a gap is defined by an intersection of the respective elliptical openings of the first and second portions; a stent graft tubular extension is provided for insertion into each of the two limbs to allow for fluid flow from the first opening of the first portion through the second and third portions and to the respective limbs and out through each of the extensions.

### BRIEF DESCRIPTION OF THE FIGURES

The foregoing and other features and advantages of the invention will be apparent from the following, more particular description of preferred embodiments of the invention, as illustrated in the accompanying drawings.

FIG. 1 illustrates a first embodiment of our invention in a semi-exploded perspective;

FIG. 1A illustrates certain parameters for coupling of the two portions with maximum overlap;

FIG. 1B illustrates certain parameters for coupling of the two portions with maximum overlap;

FIG. 1C illustrates an arterial branching stent-graft or a bridging stent-graft;

FIG. 2 illustrates a second embodiment of our invention in a semi-exploded view;

FIG. 3 illustrates a third embodiment of our invention in a semi-exploded view;

FIG. 4 illustrates the limb extensions that can be used to extend the flow passage of the bifurcated limbs of FIGS. 1-3;

FIG. 5 is a prototype of a fourth embodiment of our invention using components selected from the first through third embodiments;

FIG. 6 illustrates the first embodiment as situated in a representation of an abdominal aorta;

FIG. 7A illustrates a delivery device for the subject invention in a simplified perspective view; and

FIG. 7B illustrates an enlarged perspective view of the distal end (the end opposite the catheter handle).

FIG. 8A illustrates a human abdominal aorta with the usual arteries branching therefrom;

FIG. 8BI illustrates a presentation of an infrarenal AAA;

FIG. 8BII illustrates a presentation of a juxtarenal AAA;

FIG. 8BIII illustrates a presentation of a pararenal AAA;

FIG. 8BIV illustrates a presentation of a suprarenal AAA;

The accompanying drawings, which are incorporated herein and constitute part of this specification, illustrate presently preferred embodiments of the invention, and, together with the general description given above and the detailed description given below, serve to explain features of the invention (wherein like numerals represent like elements).

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±50% of the recited value, e.g. “about 50%” may refer to the range of values from 51% to 99%. In addition, as used herein, the terms “patient,” “host,” “user,” and “subject” refer to any human or animal subject and are not intended to limit the systems or methods to human use, although use of the subject invention in a human patient represents a preferred embodiment. The uses of the terms “cranial” or “caudal” are in this application are used to indicate a relative position or direction with respect to the person receiving the implant. As applied to “cranial,” the term indicates a position or direction closer to the heart, while the term “caudal” indicates a position or direction further away from the heart of such a subject.

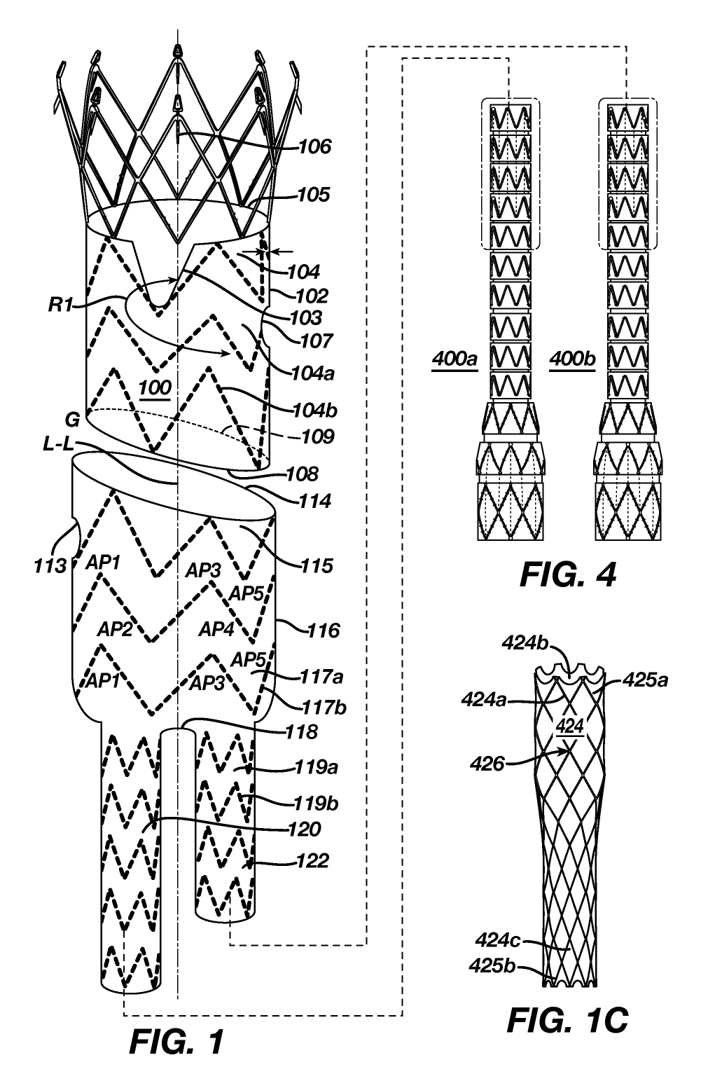

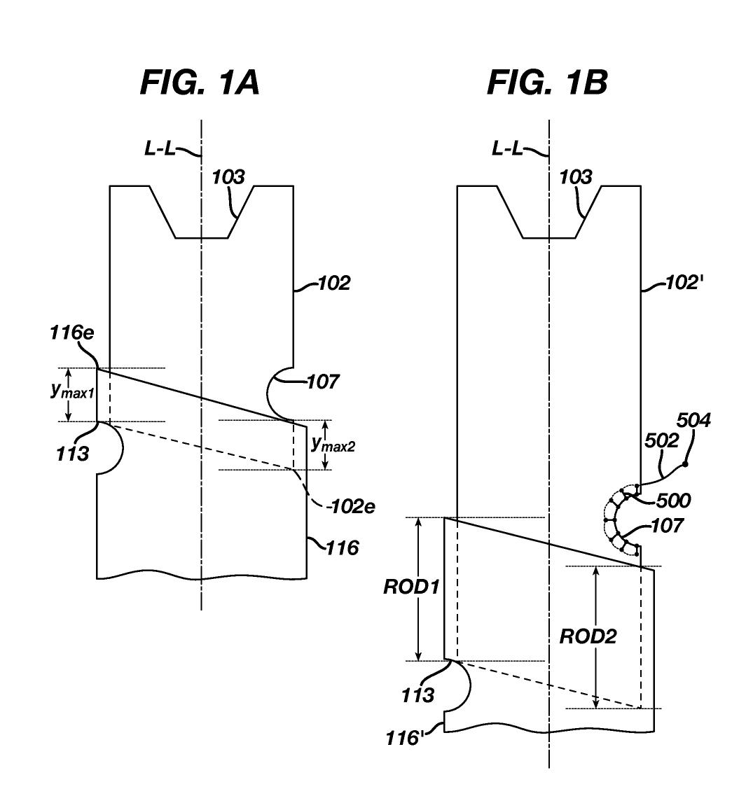

A first embodiment of an endovascular implant (100) is shown in FIG. 1 that can be used with limb extensions in EVAR procedures for AAAs that are other than infra-renal. In other words, the implant 100 can be used in AAA that categorized as juxtarenal, pararenal or suprarenal type AAAs due to its particular configuration. In particular, the implant 100 is formed as a combination of three main elements: (a) a first tubular like first portion, (b) a second tubular like portion with a bifurcation, and (c) a retainer section for the first or second sections. The first portion 102 extends along a longitudinal axis L-L. The first portion 102 includes a suitable graft material in the form of a generally tubular graft 104a. The graft 104a is configured to define a generally circular opening 105 orthogonal to the longitudinal axis L-L. In this particular embodiment, retention structure is utilized to connect retention barbs 106 to the generally circular opening 105. The first portion 102 includes a second end 108 with the graft material defining a generally elliptical opening 109 about the longitudinal axis L-L. The elliptical opening 109 can be formed by sectioning the generally tubular graft material at angle other than 90 degrees with respect to the longitudinal axis L-L.

Continuing in FIG. 1, there is provided a second portion 116 that extends along the longitudinal axis L-L. In this second portion 116, a first end 115 is provided by a suitable graft material in the form of a generally tubular graft 104a. Tubular graft 104a defines a generally elliptical opening 114 with respect to the longitudinal axis L-L to allow the second end 108 of the first portion 102 to extend into the generally elliptical opening 114 of the second portion 116 (or vice versa—the elliptical opening 114 extending into the second end 108 of the first portion). As noted earlier, the second portion 116 has a bifurcation 118 that extends into two limbs 120, 122 which can be configured to extend along the longitudinal axis.

Each of the limbs can be connected to respective extensions 400a and 400b, shown here in FIG. 4. The limb extensions 400a and 400b allow blood to flow through the first section 102, second section 116 into the limbs 120 and 122 then into respective extensions 400a and 400b which are implanted into the respective left and right common iliac arteries.

The constructions of each of the sections and extensions are very similar. In particular, each of the first and second portions 102, 116 may include a plurality of stent hoops (104b or 204b) spaced along the longitudinal axis L-L and attached to a graft material by a suitable technique (e.g., suture, weave or bonding) to define a composite stent graft in the form of implant 100. Each of the stent hoops 204b may have a sinusoidal configuration disposed about the longitudinal axis with apices AP1, AP3, AP5 . . . APn spaced apart with respect to apices AP2, AP4, AP6 . . . APn+1 (where n is an odd integer including zero) along the longitudinal axis. It is noted here that one apex (e.g., AP2) of one stent hoop is disposed between two apices (e.g., AP1 and AP2) of another stent hoop. In the preferred embodiments, the graft material may be a synthetic material selected from a group consisting of nylon, ePTFE, PTFE, Dacron and combinations thereof. Preferably, the plurality of stent hoops 204b are disposed on the inside surface of the stent-graft including that of the limb extensions. Details of the constructions of the stent hoops and graft materials are shown and described in U.S. patent application Ser. No. 14/316,151 filed on Jun. 26, 2014 (Attorney Docket No. CRD5524USNP), which is hereby incorporated by reference into the application.

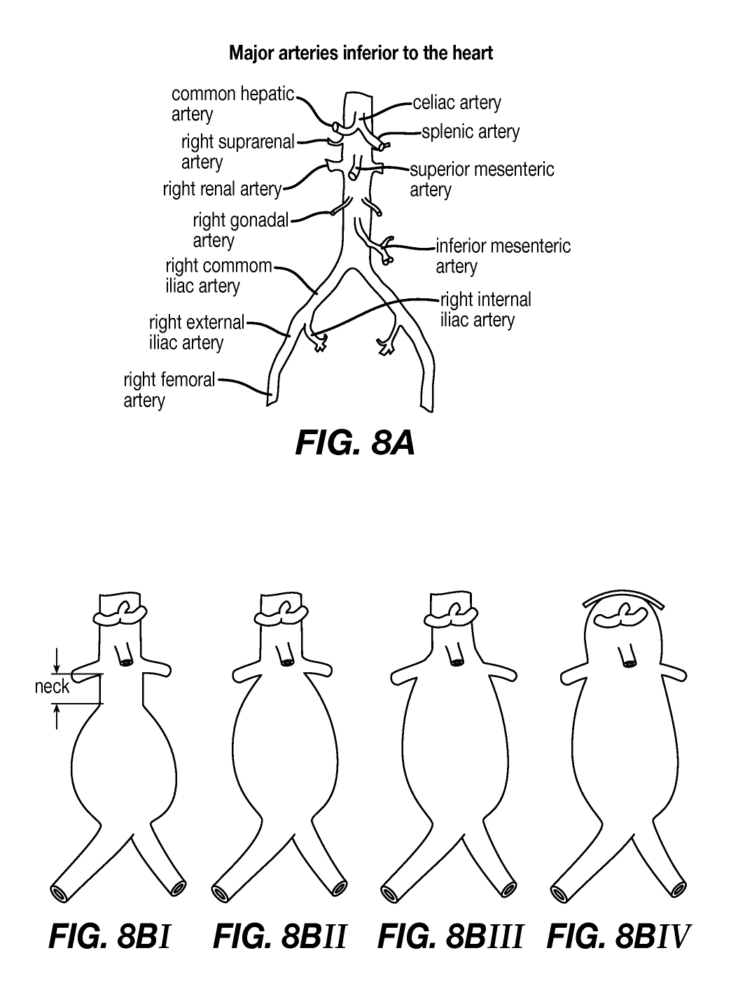

In FIG. 1, a first peripheral opening 103 is formed through the graft 104a material about the longitudinal axis of the first portion 102 proximate the first end 104 and so that the first peripheral opening 103 faces a mesenteric artery (e.g., superior mesenteric artery in FIG. 8A) when the implant 100 is deployed in an abdominal artery. A second peripheral opening 107 is formed through the graft 104a material about the longitudinal axis of the first portion 102 so that the second peripheral opening 107 faces a renal artery (FIG. 8A) when the implant 100 is deployed in the abdominal artery. On the second section 116, another peripheral opening 113 is formed through the graft 104a material about the longitudinal axis of the second portion 116 so that the peripheral opening 113 faces another renal artery when the implant 100 is deployed in the abdominal artery.

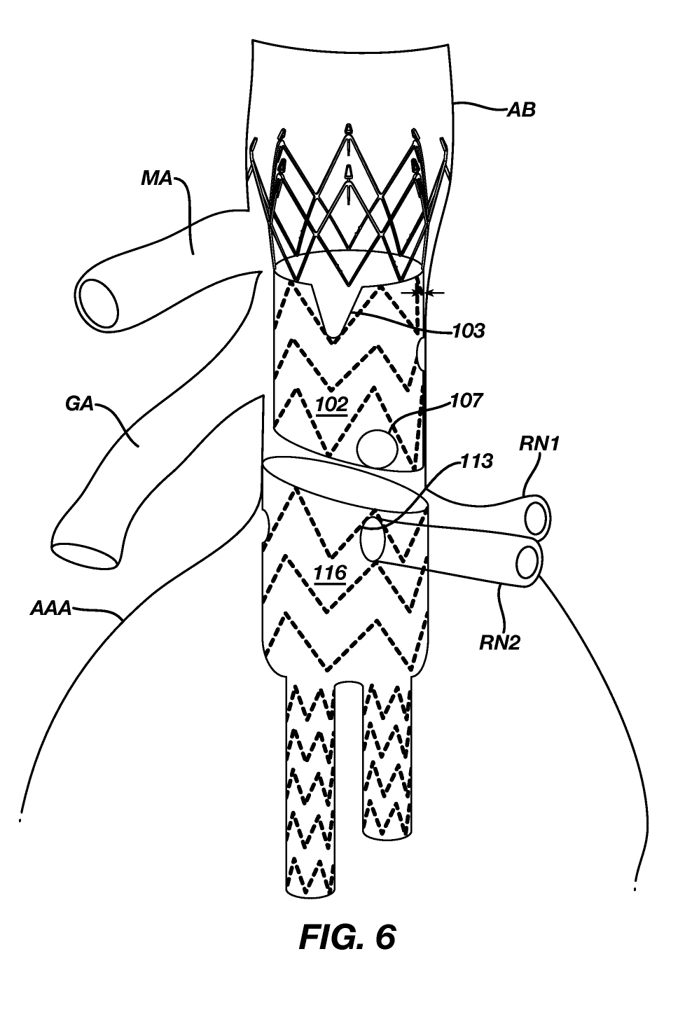

By virtue of our design, we are able to account for variations in the biological anatomies in where the renal arteries are oriented with respect to the abdominal aorta connected to the heart. Referring to FIG. 6, it can be seen that the first portion 102 is radially adjustable (i.e., rotatable about the longitudinal axis L-L as indicated by “R1”) in the abdominal aorta AB with respect to the second portion 116 so that the first peripheral opening 107 on the first portion 102 is oriented correctly to the infrarenal arteries RN1 and RN2. In most cases, the first peripheral opening 107 is oriented so that it is generally diametrical to the peripheral opening 113 on the second portion 116. Regardless of whether the infrarenal arteries are diametrical, a gap G is defined by the intersection of the respective elliptical openings 109, 114 of the first and second portions 102, 116 to allow for blood flow to the right gonadal artery GA or the inferior mesenteric artery MA.

Referring to FIG. 1A, there is one consideration that should be followed for use of the exemplary implant. Specifically, the peripheral opening (113 or 107) should be spaced at a maximum distance (yₘₐₓ₁ or yₘₐₓ₂) from the furthest point (116e or 102e) to the circumferential edge of the graft (116 or 102 respectively) on which the peripheral opening is formed therein. For example, peripheral opening 113 should have a maximum distance of yₘₐₓ₁ (as measured parallel to axis L-L) from the furthest point 116e on the circumferential opening of graft 116. Similarly, peripheral opening 107 should have a maximum distance of yₘₐₓ₂ (as measured parallel to axis L-L) from the furthest point 102e on the circumferential edge of the graft 102 to ensure a good seal between the graft with the abdominal artery. It is noted that the minimum overlap distance is the smallest longitudinal distance of the overlap between the two portions sufficient to ensure a good seal between the two implant portions. Another consideration is the longitudinal offset distance (along axis L-L) between the two renal arteries (i.e., “renal offset distance”). By virtue of our invention, a given range of renal offset distance can be covered by an implant with a given maximum overlap. As an example, a renal offset distance of 5 millimeters can be used with an implant with a yₘₐₓ of 10 mm whereas a renal offset of 12 mm would require an implant with a greater yₘₐₓ. Thus, there are instances, depending on the peculiarity of the actual anatomy that may require a clinic to keep an inventory of a number of implants with different renal offset distances. For example, a clinic may keep first stock keeping unit (“SKU”) similar to FIG. 1A that has a minimum renal offset distance of about 5 mm along with a range of renal offset distances (in a stepped gradation of for example 2 mm) up to an SKU in the configuration of FIG. 1B. In FIG. 1B, the implants are configured to have a renal offset distance ROD1>yₘₐₓ₁ and ROD2>yₘₐₓ₂. In the preferred embodiments, each of the longitudinal overlap distances yₘₐₓ₁ or yₘₐₓ₂ is about 5 millimeters and renal offset distances ROD1 or ROD2 may be up to about 40 millimeters and most preferably, each of ROD1 or ROD2 is from about 5 millimeters to about 25 millimeters.

While the two peripheral openings are shown as being diametrically opposed when the farthest point (116e) on the circumference of one graft (116) is aligned with the farthest point (102e) on the circumference of the ellipse for the other graft (102), it should be noted that many different radial orientations (with respect to axis L-L) can be provided depending on the peculiarity of the arterial anatomy being presented. In most cases, it is believed that the illustrated arrangement (FIG. 1A) of the peripheral openings 113 and 107 with approximately 150 degrees offset is sufficient as a first SKU for most cases of AAA. Nevertheless, additional SKUs can be provided for other range of angular separation (e.g., 30-90 degrees with respect to each other as referenced to the L-L axis) as appropriate.

Referring to FIG. 1C, an arterial stent graft extension (or bridging stent, as known in the field) 424 can be used for insertion into peripheral openings 104 and 130 so that side arteries can be incorporated into the flow of the implant. The extension 424 has a suitable biocompatible graft material 424a similar to the graft material of the main portions noted earlier. The extension is configured as a generally tubular flow through structure. In one embodiment, the extension 424 has a generally circular opening 424b at one end 425a. The extension 424 tapers from the first end 425a towards a smaller second generally circular extension opening 424c proximate the other end 425b. The arterial stent graft extension 424 is configured for insertion into at least one of the peripheral openings of the first and second portions with retainers provided proximate each end 425a and 425b to retain the extension to the main portions of the implant or the blood vessel. The arterial extension or bridging stent-graft 424 may have at least one stent hoop 426 expandable to support the arterial stent graft 424a. Alternatively, the stent hoop 426 can be a plurality of separate stent hoops connected to each other via the graft material for extension 424.

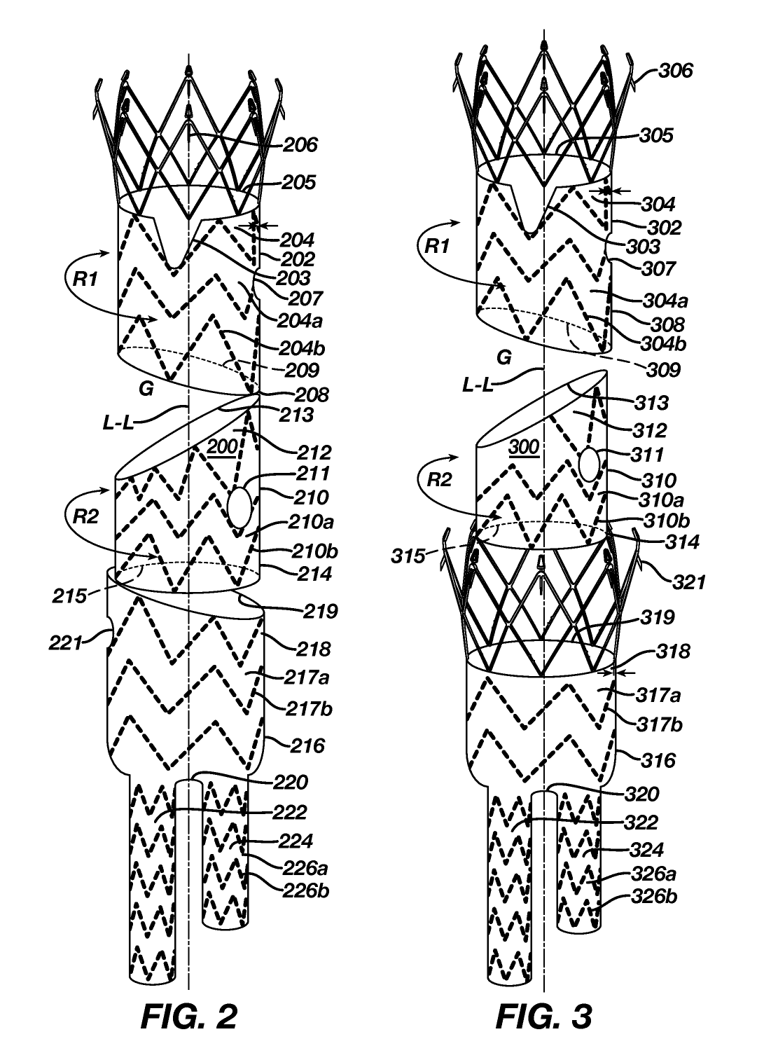

Another embodiment of our inventive implant is shown in FIG. 2. In FIG. 2, there are four main components to implant 200: (a) a cranial or first tubular like section; (b) an intermediate tubular like section 210; (c) a caudal tubular like section that bifurcates into two limbs; and (d) a retention structure to assist in retaining one or both of the cranial and caudal sections.

Starting with the first portion 202 which extends along a longitudinal axis L-L, this section of the implant 200 may include a first end 204 defining a generally circular opening 205 orthogonal to the longitudinal axis L-L. A retention structure with retention barbs 206 may be provided. The retention structure extends from the generally circular opening 205 so as to be coupled to the retention barbs 206. Moving downward along the longitudinal axis L-L, the first portion 202 has a second end 208 defining a generally elliptical opening 209 about the longitudinal axis L-L. The elliptical opening 209 can be sectioned from the tubular structure at an angle other than 90 degrees to the longitudinal axis so as to provide a suitable size ellipse.

Turning to the second portion 210, this portion of the implant 200 extends along the longitudinal axis L-L for a suitable length with a first end 212 defining a generally elliptical opening 213 with respect to the longitudinal axis L-L. The elliptical opening 213 formed into the tubular structure allows the second end 208 of the first portion 202 to extend into the generally elliptical opening 213 of the second portion 210 while still leaving a gap G between the two sections. Moving downward along the longitudinal axis, the second portion 210 also has a second end 214 defining a generally circular opening 215 orthogonal to the longitudinal axis L-L.

A third portion 216 is provided which extends along the longitudinal axis L-L. The third portion 216 has a first end 218 defining a generally elliptical opening 219 with respect to the longitudinal axis L-L. Again, this elliptical opening 219 allows the second end 214 of the second portion 210 to telescope thereto with respect to the generally elliptical opening 219 of the third portion 216 while still leaving a gap G between the sections 210 and 216 so as to allow blood flow to the appropriate arterie(s). It is noted that the third portion 216 has a bifurcation 220 that extends into two separate limbs 222, 224 extending along the longitudinal axis L-L.

The constructions of each of the sections and extensions in this second embodiment are very similar to the first embodiment shown in FIG. 1 and described earlier. Each of the first, second and third portions 202, 210, 216 has a plurality of discrete stent hoops 204b, 210b, 226b spaced along the longitudinal axis L-L and attached to a graft material 204a, 210a to define a stent-graft composite endoprosthesis 200. As noted earlier, each of the stent hoops 204b, 210b, 226b having a sinusoidal configuration disposed about the longitudinal axis L-L with apices of each stent hoop spaced apart along the longitudinal axis with respect to the apices of adjacent stent hoops. As in the first embodiment, the apex of one stent hoop is disposed between two apices of an adjacent stent hoop(s); the generally tubular graft 204a, 210a, 226a may include a synthetic material selected from a group consisting of nylon, ePTFE, PTFE, Dacron and combinations thereof; and preferably, the plurality of stent hoops 204b, 210b, 226b are disposed on the inside surface of the stent-graft.

For fluid flow incorporation of the appropriate artery, a first peripheral opening 203 is formed through the graft material 204a of the first section 202 and about the longitudinal axis of the first portion 202 proximate the first end 204 and so that the peripheral opening 203 faces a mesenteric artery when the implant 200 is deployed in an abdominal artery (FIG. 8A).

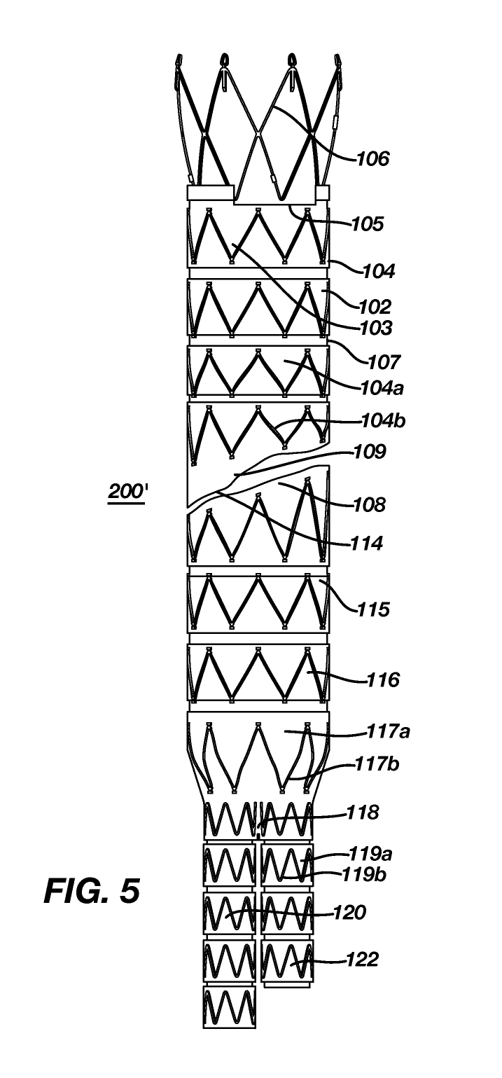

Similarly, for blood flow incorporation of a renal artery, a second peripheral opening 207 is formed through the graft material 204a radially about the longitudinal axis of the first portion 202 so that the peripheral opening 207 faces a renal artery when the implant 200 is deployed in the abdominal artery. Likewise, for incorporation of the other renal artery, another peripheral opening 211 is formed through the graft material 204a, 210a radially about the longitudinal axis L-L of the second portion 210 so that the peripheral opening 211 faces another renal artery when the implant 200 is deployed in the abdominal artery. It is preferable that the fenestrations or peripheral openings be disposed in the open space between the longitudinally spaced-apart sinusoidal like stent hoops. It is noted that the stent hoops are preferably sinusoidal (FIGS. 1 and 5) but do not have to be and therefore can be in an irregular zig-zag configuration (FIGS. 2 and 3). For example, as can be seen in FIGS. 2 and 3, the stent hoop proximate the elliptical openings 209 and 213 (as well as 309 and 313) are irregular sized zig-zag stent to allow for the bevel-like configuration of these openings to be formed.

To allow alignment of the peripheral openings with the respective arteries (renal in this case), the first and second portions 202 and 210 can be rotated about the longitudinal axis as indicated by arrows R1. That is, the first portion 202 is radially adjustable (reference arrow R1) with respect to the second portion 210 so that the first peripheral opening 107 on the first portion 202 can be generally diametrical to the peripheral opening 211 on the second portion 210 and a gap G is defined by an intersection of the respective elliptical openings 209, 213 of the first and second portions 202 and 210.

FIG. 5 shows implant 200′ as a variation of the implant 200 of FIG. 2 in which the same reference numbers in FIG. 5 indicate the same elements in FIG. 2. In this variation, we have devised the elliptical openings 109 and 114 of the respective sections so that they are orientated differently with respect to the peripheral openings 103 and 107. This illustrates one of the benefits of our design in allowing for customization of the implant without the device having to be made tediously by hand.

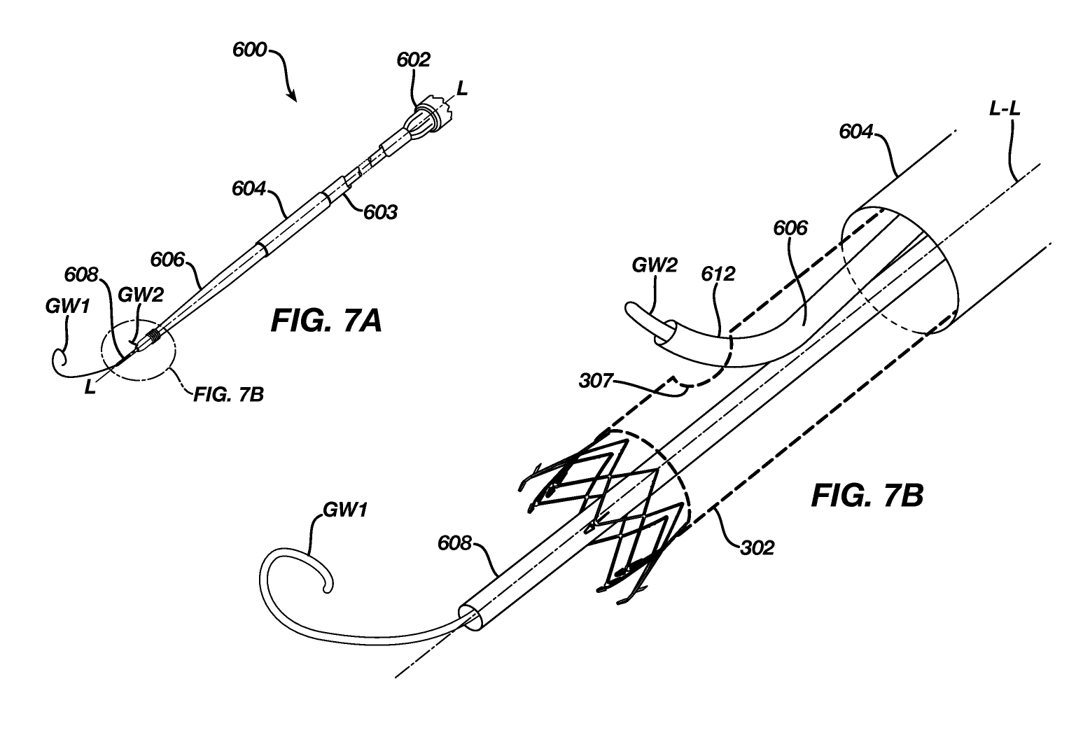

As noted earlier to the first embodiment of FIG. 1, limb extensions 400a and 400b can be utilized to allow the flow of blood through the first, second, and third sections into the limbs and limb extensions into the respective iliac arteries. In most instances, each of the peripheral openings (107, 113 in FIG. 6A) usually forms a sufficient conduit from the abdominal artery to the branching artery such that no bridging stent is needed. Nevertheless, there are instances when bridging stent grafts 124 may be utilized to prevent leakage. In such instances, the bridging stent graft 324 can be used to connect the peripheral openings (e.g., 211) on the graft to the branch arteries (RN1 and RN2) in FIG. 7.

Referring to FIG. 3, yet a third variation of the inventive prosthesis is shown. In this variation, there is an additional retention mechanism (with barbs 321) for the caudal portion 316 of the implant 3000. In other respects, the structures of implant 300 are substantially the same as that of implant 200. Nevertheless, for the sake of completeness, implant 300 is described in detail below.

Implant 300 includes a first portion 302 that extends along a longitudinal axis L-L. The first portion 302 has a first end 304 defining a first generally circular opening 305 orthogonal with respect to the longitudinal axis L-L. A scaffold in the form of a diamond shaped stent is attached to a terminal end i.e., the generally circular opening 305 of the first end of the first portion. The scaffold is provided with retention barbs 306 extending at an angle with respect to the longitudinal axis. The first portion 302 includes a second end defining a generally elliptical opening about the longitudinal axis L-L.

The second portion 310 extends along the longitudinal axis L-L and has a first end 304 defining a generally elliptical opening 312 with respect to the longitudinal axis L-L. This elliptical opening 312 allow the second end 308 of the first portion 302 to extend into the generally elliptical opening 313 of the second portion 310, the second portion 310 having a second end 314 defining a second generally circular opening 315 orthogonal to the longitudinal axis L-L. The third portion extends along the longitudinal axis L-L and has a first end 304 defining a third generally circular opening orthogonal to the longitudinal axis L-L to allow the second end of the second portion to telescope with respect thereto the first end 304 of the third portion. Note that the first end 304 of the third portion has retention members 321 coupled to the third generally circular opening 319 via a diamond shaped stent. The third portion 316 has a bifurcation 320 that extends into two limbs extending along the longitudinal axis L-L.

Each of the first, second and third portions may include a plurality of stent hoops spaced along the longitudinal axis L-L and attached to a graft material to define a stent graft composite implant 100. Each of the stent hoops has a generally sinusoidal configuration disposed about the longitudinal axis L-L. The apices of such stent hoop are spaced apart along the longitudinal axis L-L with respect to the apices of adjacent but separate stent hoops. As used herein, the term “separate” in relation to the stent hoops means that the hoops are not connected with connectors that are made of the same material as the hoop but via a different material. To allow for a thin profile prior to deployment, i.e., less than 12 French (and in some cases where the native artery is large, the implant pre-deployment profile can be less than 16 French), we have devised the stent hoop so that one apex of one stent hoop is disposed between two apices of another stent hoop shown as an example in FIG. 1. The graft material for the tubular sections can be any suitable material but generally may include a synthetic material selected from a group consisting of nylon, ePTFE, PTFE, Dacron and combinations thereof. While the stent hoops can be on the outside surface of the graft so that the hoops are in direct contact with the aorta, we prefer to have the stent hoops disposed on the inside surface of the graft or stent-graft material to prevent direct physical contact of the stent hoop to the aorta.

Similar to the first and second embodiments, a first peripheral opening 303 is formed through the graft material about the longitudinal axis of the first portion 302 proximate the first end 304 and so that the first peripheral opening 303 faces a mesenteric artery when the implant is deployed in an abdominal artery. Likewise, a second peripheral opening 307 is formed through the graft material about the longitudinal axis of the first portion 302 so that the second peripheral opening 307 faces a renal artery when the implant is deployed in the abdominal artery to allow fluid communication from the renal artery to the second peripheral opening 307. We have also devised a third peripheral opening 311 formed through the graft material about the longitudinal axis of the second portion 310 so that the third peripheral opening 311 faces another renal artery when the implant is deployed in the abdominal artery to allow fluid communication from the renal artery to the third peripheral opening 311.

By virtue of its construction, the first portion 302 is radially adjustable R1 about the longitudinal axis L-L with respect to the second portion 310 so that the first peripheral opening 303 on the first portion 302 is generally diametrical to the first peripheral opening 303 on the second portion and a gap G is defined by an intersection of the respective elliptical openings 309, 313 of the first and second portions 302 and 310.

As in the previous embodiments, a stent graft tubular extension 400a, 400b is provided for insertion into each of the two limbs to allow for fluid flow from the first opening 305 of the first portion 302 through the second and third portions 302, 310, 316 and to the respective limbs 322, 324 and out through each of the extensions 400a, 400b.

It is noted that while the peripheral openings are illustrated as circular openings formed on the circumference of the implant, other shape and configurations can be utilized that are within the scope of the invention. For example, the peripheral opening may be in the shape of a truncated circular cone that tapers towards a smaller diameter as the conic peripheral opening extends away from the longitudinal axis. Alternatively, ridges or retention ribs may be provided on the circumference of the tapered cone to allow the ribs or ridges to retain the tapered conic peripheral opening in the inner surface of the branch artery without the use of a bridging stent-graft 424.

The peripheral openings or fenestrations can be configured with sutures 500 threaded on the circumference of the fenestration 107 (FIG. 1B) to provide for an initial small opening. Extra length 502 of the suture 500 can be provided at the end of the suture 500 to provide for slack built into the suture such that when the opening 107 is dilated, the slack 502 in the suture allows for the fenestration to enlarge for matching of side branch arteries of different diameters to the fenestration 107. The suture 500 can be configured with a predetermined slack length 502 to a lock stitch 504 to prevent over dilation of the peripheral opening 107. In addition to suture 500, reinforcement in the form of another type of suture can also be provided on the circumference of the peripheral opening 107. Radiopaque markers can be disposed on the circumference of the peripheral opening (or interwoven into the suture 500) so that the physician can visualize the actual size of the fenestration 107. The peripheral openings can be dilated to the intended size in-situ (in the native artery) by insertion of a suitable dilation balloon catheter guided to the fenestration via guidewire GW2 (FIG. 7B). Upon reaching the fenestration, the balloon can be inflated gradually while being monitored via the markers of the fenestration.

In operational deployment, the surgeon is able to select from among different components described and shown exemplarily herein instead of physically making customized fenestrations on existing designs. The first portion is typically deployed first so that it forms a foundation on which to mount the remaining components. Thereafter, the second, third or even a fourth portion can be deployed in turn with the limb extensions being last. Where the AAA is presented as a juxtarenal type, the device in FIG. 1 or FIG. 2 can be utilized and each of the separate first through third portions can be rotated to achieve the desired incorporation of the arteries in the body.

Referring to FIG. 7A, a delivery device 600 is shown in perspective view with a portion of the handle 602 proximal to the operator for manipulation by the operator is shown for brevity. Attention should be directed to the distal portion 603 which is provided with an outer sheath 604, a fenestration tube 606 that allows for an inner sheath 608 to pass through while being guided by a first guide wire GW1. The fenestration tube 606 also allows the implant (first or second portions) to be mounted so that the peripheral opening of the implant can be fixed to the fenestration tube 606 such that rotation of the fenestration tube 606 allows the peripheral opening of the graft implant to be aligned to the desired branch artery. In order to explain this unique aspect of the fenestration tube, reference is made to FIG. 7B, which is an enlarged perspective view of the distal end of the delivery device 600.

In FIG. 7B, the outer sheath 604 surrounds the outer surface of graft 302 while the inner surface of the graft 302 surrounds substantially the outer surface of the fenestration tube or sheath 606. The fenestration sheath 606 is substantially parallel to the outer surface of an inner sheath 608 which can pass through the outer sheath 604. To ensure proper alignment of the peripheral opening 307 of implant 302, the fenestration tube 606 is provided with a fenestration nub 612 on which the peripheral opening 307 is fitted over, all the while the implant being radially compressed in its pre-delivery profile. It should be noted here that while examples of the implant are shown in FIGS. 1-7 in its larger deployed profile (with the attendant large outside diameter conforming against the inner surface of an abdominal artery), the implant in its pre-delivery profile is compressed into the sheath 604 to a much smaller constrained profile (with a smaller outside diameter, as small as 14 French or less).

As is known in the art, the stent graft implant (e.g., implant 302) is moved to its intended location proximate the aneurysm by way of the inner sheath 608 following the first guide wire GW1. Once the implant 302 has arrived proximate the desired site, the outer sheath 604 can be pulled back (or the implant can be pushed out of the sheath 604) to expose the fenestration nub 612. This allows a second guide wire GW2 to be pushed out of the nub 612 via a lumen provided in the fenestration tube 606 (or in another lumen built into the inner sheath 608. Under an appropriate guidance technique (e.g., fluoroscopy), the second guide wire GW2 can be manipulated (via translation or rotation of fenestration tube 606 about its longitudinal axis L-L) so that guide wire GW2 can enter into an arterial branch (e.g., a renal artery RN1 or RN2 in FIG. 6). Insertion of the second guidewire GW2 into the arterial branch will ensure that the peripheral opening (e.g., 307) will adequately mate to the arterial branch. Where desired, the second guide wire can be utilized for insertion of the arterial extension or bridging stent. Thereafter, the other implant portion(s) (e.g., 102, 202, 204, 304, or 310) can be inserted into the desired position along the first guidewire GW1 and deployed so that the other implant portion(s) can be coupled to the first implant portion.

Details of the handle and the procedures used for deployment of a similar AAA graft are shown and described in the Instruction for Use of the InCraft AAA implant (available in Europe), attached hereto the appendix. Where the AAA is presented other than an infrarenal AAA, the delivery device used for deployment can be via the device shown and described in US Patent No. U.S. Pat. No. 8,771,333, US Patent Application Publication Nos. US20070156224 and US20130085562, which are incorporated by reference as if set forth herein. It is noted that the examples provided are initially intended for AAAs, applications for other arterial sites with branching arteries can also be utilized such as, for example, in a thoracic aortic aneurysm or TAA where angulation of the artery may cause difficulty in forming a tight seal between the artery and the graft.

All of the stent hoops described herein are substantially tubular elements that may be formed utilizing any number of techniques and any number of materials. In the preferred exemplary embodiment, all of the stent hoops are formed from a nickel-titanium alloy (Nitinol), shape set laser cut tubing.

The graft material utilized to cover all of the stent hoops may be made from any number of suitable biocompatible materials, including woven, knitted, sutured, extruded, or cast materials forming polyester, polytetrafluoroethylene, silicones, urethanes, and ultra-light weight polyethylene, such as that commercially available under the trade designation SPECTRA™. The materials may be porous or nonporous. Exemplary materials include a woven polyester fabric made from DACRON™ or other suitable PET-type polymers.

As noted above, the graft material is attached to each of the stent hoops. The graft material may be attached to the stent hoops in any number of suitable ways. In the exemplary embodiment, the graft material is attached to the stent hoops by sutures.

Depending on the stent hoops location, different types of suture knots may be utilized. Details of various embodiments of the suture knots for suture can be found in US Patent Application Publication No. US20110071614 filed on Sep. 24, 2009, which is hereby incorporated by reference as if set forth herein.

While the invention has been described in terms of particular variations and illustrative figures, those of ordinary skill in the art will recognize that the invention is not limited to the variations or figures described. For example, while examples are shown for AAA, these implants can also be utilized for thoracic aortic aneurysm (TAA), which may not require retention barbs for use in TAA. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An endovascular implant comprising:
a first portion extending along a longitudinal axis, the first portion comprising a generally tubular graft defining a generally circular opening about the longitudinal axis, the first portion including a second end defining a generally elliptical opening about the longitudinal axis; and
a second portion extending along the longitudinal axis, the second portion having a first end including a generally tubular graft defining a generally elliptical opening with respect to the longitudinal axis to allow the second end of the first portion to extend into the generally elliptical opening of the second portion, the second portion having a bifurcation that extends into two limbs extending along the longitudinal axis.

2. The endovascular implant of claim 1, in which each of the first and second portions comprises a plurality of stent hoops spaced along the longitudinal axis and attached to a graft material to define a stent graft composite implant, each of the stent hoops having a sinusoidal configuration disposed about the longitudinal axis with apices spaced apart along the longitudinal axis.

3. The endovascular implant of claim 2 in which one apex of one stent hoop is disposed between two apices of another stent hoop.

4. The endovascular implant of claim 2 in which the generally tubular graft comprises a synthetic material selected from a group consisting of nylon, ePTFE, PTFE, Dacron and combinations thereof.

5. The endovascular implant of claim 2 in which the plurality of stent hoops are disposed on the inside surface of the stent-graft.

6. The endovascular implant of claim 2 in which a first peripheral opening is formed through the graft material about the longitudinal axis of the first portion proximate the first end and so that the first peripheral opening faces a mesenteric artery when the implant is deployed in an abdominal artery.

7. The endovascular implant of claim 6, in which a second peripheral opening is formed through the graft material about the longitudinal axis of the first portion so that the second peripheral opening faces a renal artery when the implant is deployed in the abdominal artery.

8. The endovascular implant of claim 7, in which a first peripheral opening is formed through the graft material about the longitudinal axis of the second portion so that the peripheral opening faces another renal artery when the implant is deployed in the abdominal artery.

9. The endovascular implant of claim 8, in which the first portion is radially adjustable with respect to the second portion so that the first peripheral opening on the first portion is generally diametrical to the peripheral opening on the second portion and a gap is defined by the intersection of the respective elliptical openings of the first and second portions.

10. The endovascular implant of claim 9, in which a tubular a tubular stent graft extension is provided for insertion into each of the two limbs to allow for fluid flow from the first opening of the first portion through the second portion to the respective limbs of the implant and out through each of the extensions.

11. The endovascular implant of claim 6, in which the peripheral opening is configured for a first smaller opening and dilatable to a second larger opening.

12. An endovascular implant comprising:
a first portion extending along a longitudinal axis, the first portion having a first end defining a generally circular opening orthogonal to the longitudinal axis with retention barbs coupled to a retention structure connected to the generally circular opening, the first portion including a second end defining a generally elliptical opening about the longitudinal axis;
a second portion extending along the longitudinal axis, the second portion having a first end defining a generally elliptical opening with respect to the longitudinal axis to allow the second end of the first portion to extend into the generally elliptical opening of the second portion, the second portion having a second end defining a generally circular opening orthogonal to the longitudinal axis; and
a third portion extending along the longitudinal axis, the third portion having a first end defining a generally elliptical opening with respect to the longitudinal axis to allow the second end of the second portion to telescope thereto with respect to the generally elliptical opening of the third portion, the third portion having a bifurcation that extends into two limbs extending along the longitudinal axis.

13. The endovascular implant of claim 12, in which each of the first, second and third portions comprises a plurality of stent hoops spaced along the longitudinal axis and attached to a graft material to define a stent graft composite implant, each of the stent hoops having a sinusoidal configuration disposed about the longitudinal axis with apices spaced apart along the longitudinal axis.

14. The endovascular implant of claim 13, in which one apex of one stent hoop is disposed between two apices of another stent hoop.

15. The endovascular implant of claim 13, in which the generally tubular graft comprises a synthetic material selected from a group consisting of nylon, ePTFE, PTFE, Dacron and combinations thereof.

16. The endovascular implant of claim 13, in which the plurality of stent hoops are disposed on the inside surface of the stent-graft.

17. The endovascular implant of claim 13, in which a first peripheral opening is formed through the graft material about the longitudinal axis of the first portion proximate the first end and so that the peripheral opening faces a mesenteric artery when the implant is deployed in an abdominal artery.

18. The endovascular implant of claim 17, in which a second peripheral opening is formed through the graft material about the longitudinal axis of the first portion so that the peripheral opening faces a renal artery when the implant is deployed in the abdominal artery.

19. The endovascular implant of claim 18, in which another peripheral opening is formed through the graft material about the longitudinal axis of the second portion so that the peripheral opening faces another renal artery when the implant is deployed in the abdominal artery.

20. The endovascular implant of claim 19, in which the first portion is radially adjustable with respect to the second portion so that the first peripheral opening on the first portion is generally diametrical to the peripheral opening on the second portion and a gap is defined by an intersection of the respective elliptical openings of the first and second portions.

21. The endovascular implant of claim 20, in which a stent graft tubular extension is provided for insertion into each of the two limbs to allow for fluid flow from the first opening of the first portion through the second and third portions to the respective limbs of the implant and out through each of the extensions.

22. An endovascular implant comprising:
a first portion extending along a longitudinal axis, the first portion having a first end defining a first generally circular opening orthogonal to the longitudinal axis with retention barbs coupled to a retention structure connected to the generally circular opening, the first portion including a second end defining a generally elliptical opening about the longitudinal axis;
a second portion extending along the longitudinal axis, the second portion having a first end defining a generally elliptical opening with respect to the longitudinal axis to allow the second end of the first portion to extend into the generally elliptical opening of the second portion, the second portion having a second end defining a second generally circular opening orthogonal to the longitudinal axis; and
a third portion extending along the longitudinal axis, the third portion having a first end defining a third generally circular opening orthogonal to the longitudinal axis to allow the second end of the second portion to telescope with respect thereto the first end of the third portion, the first end of the third portion having retention members coupled to the third generally circular opening, the third portion having a bifurcation that extends into two limbs extending along the longitudinal axis.

23. The endovascular implant of claim 22 in which each of the first, second and third portions comprises a plurality of stent hoops spaced along the longitudinal axis and attached to a graft material to define a stent graft composite implant, each of the stent hoops having a sinusoidal configuration disposed about the longitudinal axis with apices spaced apart along the longitudinal axis.

24. The endovascular implant of claim 23, in which one apex of one stent hoop is disposed between two apices of another stent hoop.

25. The endovascular implant of claim 23, in which the generally tubular graft comprises a synthetic material selected from a group consisting of nylon, ePTFE, PTFE, Dacron and combinations thereof.

26. The endovascular implant of claim 23, in which the plurality of stent hoops are disposed on the inside surface of the stent-graft.

27. The endovascular implant of claim 23, in which a first opening is formed through the graft material about the longitudinal axis of the first portion proximate the first end and so that the first opening faces a mesenteric artery when the implant is deployed in an abdominal artery.

28. The endovascular implant of claim 27, in which a second opening is formed through the graft material about the longitudinal axis of the first portion so that the second opening faces a renal artery when the implant is deployed in the abdominal artery to allow fluid communication from the renal artery to the second opening.

29. The endovascular implant of claim 28, in which a third opening is formed through the graft material about the longitudinal axis of the second portion so that the third opening faces another renal artery when the implant is deployed in the abdominal artery to allow fluid communication from the renal artery to the third opening.

30. The endovascular implant of claim 29, in which the first portion is radially adjustable with respect to the second portion so that the first opening on the first portion is generally diametrical to the first opening on the second portion and a gap is defined by an intersection of the respective elliptical openings of the first and second portions.

31. The endovascular implant of claim 30, in which a stent graft tubular extension is provided for insertion into each of the two limbs to allow for fluid flow from the first opening of the first portion through the second and third portions and to the respective limbs and out through each of the extensions.

32. A delivery device for deployment of an implant in a body artery, the device having a handle portion and a delivery portion distal to the handle, the delivery portion including:
a first guide wire extending from the delivery portion to the handle portion;
an inner tube in which the first guide wire extends through the inner tube to the handle portion;
a fenestration tube disposed generally parallel to the inner tube and configured to be surrounded by at least a portion of an inner surface of the implant, the fenestration tube includes a fenestration nub coupled to a peripheral opening provided through the inner and outer surfaces of the implant;
an outer sheath surrounding at least a portion of the outer surface of the implant so that the implant is constrained to an outer profile smaller than a deployed outer profile.

33. The delivery device of claim 32, further comprising a second guidewire configured for movement through a lumen provided in the fenestration tube to an opening in the fenestration nub so that the second guidewire is guided for entry into an arterial branch of a major artery upon translation and rotation of the fenestration tube about its longitudinal axis.
